(19) United States
(12) Patent Application Publication
(10) Pub. No.: US 2012/0156136 A1
(43) Pub. Date: Jun. 21, 2012

(54) METHOD AND DEVICE FOR IDENTIFYING A MATERIAL LIKELY TO GENERATE EYE OR SKIN DISCOMFORT, TINGLING, OR IRRITATION

(76) Inventor: Didier Neuzeret, Sainte Euphemie (FR)

(21) Appl. No.: 13/393,181

(22) PCT Filed: Aug. 31, 2010
(86) PCT No.: PCT/EP10/62720

§ 371 Date: Feb. 28, 2012

(30) Foreign Application Priority Data
Sep. 2, 2009 (FR) 0955968

(51) Int. Cl. A61K 49/00

(52) U.S. Cl. 424/9.1

(57) ABSTRACT

The invention relates to a method for identifying a product capable of generating eye or skin discomfort, characterized in that it comprises the steps:
deposition (10) of a product in a container,
placement of an earthworm in the container,
measurement (20, 30, 40) of the movement of the earthworm in the container,
determination (50, 60) of eye discomfort which may be generated by the product according to said measurement.

The present invention relates to the general technical field of methods and devices with which it is possible to identify whether a product is able to generate discomfort, tingling or eye or skin irritation.

This product is for example a pharmaceutical product—such as a collyium—or a cosmetic product, such as a shampoo, a skincare, cream etc—or any other product, for example a chemical, which may be in contact with the skin of an individual.

### PRESENTATION OF THE PRIOR ART

A method is known for estimating eye discomfort of a product such as a cosmetic product.

This method comprises the in vitro culture of an epidermis model, the deposition of the product which is intended to be studied on a cultivated epidermis model, and the observation under the microscope of the epidermis model including the product for characterizing the eye discomfort of the latter.

Such a method, developed as a replacement for animal experimentation, has the drawback of being long and costly.

Moreover, with this method it is not possible to obtain satisfactory results in terms of quantification of the eye discomfort of the product.

An object of the present invention is to propose a method and a device providing a better estimation of eye discomfort of a product at a lesser cost.

### SUMMARY OF THE INVENTION

For this purpose, the invention proposes a method for identifying a material likely to generate eye or skin discomfort, tingling or irritation, wherein the method comprises the steps:
deposition of a product in a container,
placement of an earthworm or nematode in the container,
measurement of the movement of the earthworm in the container,
determination of eye discomfort which may be generated by the product according to said measurement.

Preferred but non limiting aspects of the method according to the invention are the following:
the step for measuring the movement of the earthworm comprises the acquisition of a plurality of images of the earthworm by using image acquisition means, and analysis of the acquired images for measuring the movement of the earthworm;
the analysis of the acquired images comprises the selection of two successive acquired images, the comparison of the luminosity of each pixel of the first image with the luminosity of the corresponding pixel in the second image, the counting of the number of pixels for which luminosity has changed between the first and second images;
the generation of a time response curve from the previous counting
the step for determining eye discomfort comprises the estimation of the irritating effect of the product and/or of the antalgic and/or anesthetic effect of the product;
the irritating and/or antalgic effect of the product is estimated according to the number of pixels, the luminosity of which has changed between successively acquired images;
if the number of pixels for which luminosity has changed between the first and second images is greater than a threshold, then the product has an irritating effect;
if the number of pixels for which luminosity has changed between the first and second images is lower than a threshold, then the product has an antalgic and/or anesthetic effect;
the determination of eye discomfort further is dependent on time;
the method comprises the illumination of the container containing the earthworm by using infrared radiation.

The invention also relates to a device for identifying a material likely to generate eye or skin discomfort, wherein the device comprises a processor programmed for applying the method described above.

The invention also relates to a computer programme product including programme code recorded on a computer readable medium which may be read by a computer for executing the method described above when the computer programme is applied to a computer in order to be executed.

### SHORT DESCRIPTION OF THE DRAWINGS

Other advantages and features will become better apparent from the following description of several alternative embodiments, given as non-limiting examples, from the appended drawings wherein:

FIG. 1 illustrates an embodiment of the method according to the invention,

FIG. 2 illustrates an embodiment of the device according to the invention,

FIG. 3 illustrates the number of pixels, the luminosity of which has changed over time,

FIG. 4 illustrates a time representation of a table indicating for a given product, the time spent in hyperactivity for a period of 60 seconds and this for a total of 10 minutes.

### DETAILED DESCRIPTION OF THE INVENTION

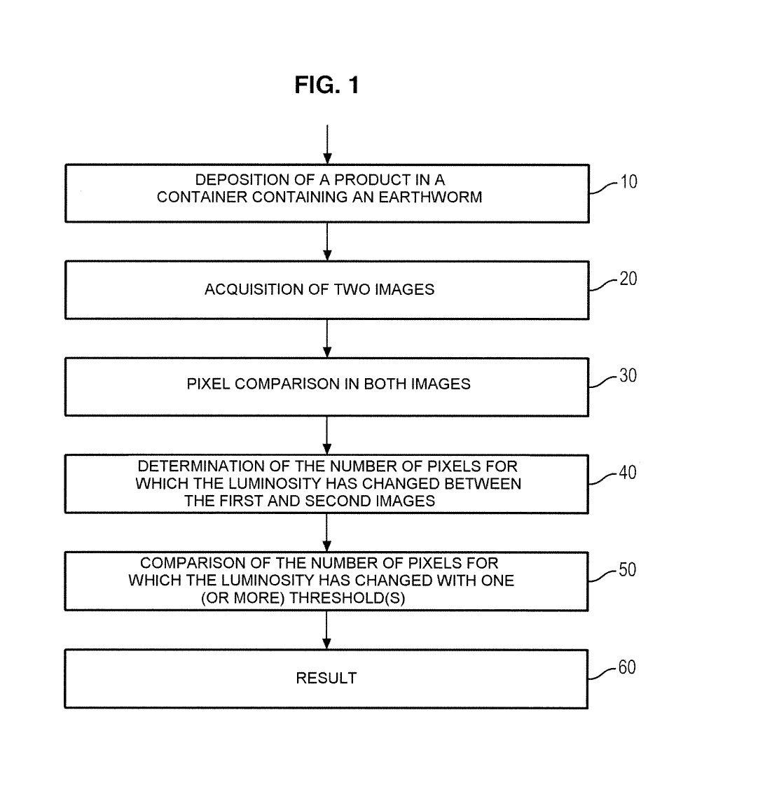

With reference to FIG. 1, an embodiment of the method according to the invention is a method with which a product which may generate discomfort, tingling or eye or skin irritation, may be identified.

The method comprises the steps:
deposition 10 of a product in a container containing an earthworm,
measurement 20, 30, 40 of the movement of the earthworm in the container,
determination 50, 60 of the eye discomfort which may be generated by the product according to this measurement.

The advantage of the use of an earthworm is that its skin has good penetration/absorption of the product which is desirably analyzed.

Indeed, depending on the movements of the earthworm in the container following the deposition of the product, the method according to the invention gives the possibility of determining whether the product is capable of generating eye discomfort.

For example, if the movements of the earthworm are highly significant following the introduction of the product, then the product has irritating properties.

If the earthworm no longer moves in the container, this may either be related to the death of the latter (if the product is highly irritating notably), or to the fact that the product has antalgic properties, which is not desirable for a product such as a collyrium, a shampoo or a cream.

The use of an earthworm within the scope of the method according to the invention therefore allows identification of whether a product is capable or not of generating eye or skin discomfort.

Preferably, the earthworm is of the “Eisenia” type.

The step for measuring the movement of the earthworm may be achieved by using different measurement systems. For example, it is possible to measure the movement of the earthworm by using a system comprising a support provided with strain gauges. The support is intended to receive the container containing the earthworm. The strain gauges allow measurement of the change of attitude of the support over time, and thus an estimation of the movement of the earthworm.

In the embodiment illustrated in FIG. 1, the step for measuring the movement of the earthworm is achieved by image analysis. The principle of the measurement of the movement of the earthworm is then the following.

A series of images is acquired 20 by acquisition means all along the experiment. The acquisition means allow acquisition of grey level images, i.e. to each pixel of an image corresponds a value—representative of luminosity, comprised between 0 (black) and 255 (white).

During the gradual acquisition (or alternatively at the end of the acquisition) the images are compared 30 two by two. Each image acquired at time t is compared with the successive acquired image at time t+1. The purpose of this comparison is to determine a number of pixels for which the luminosity has changed between both images.

To do this, in an embodiment of the invention, the second image is subtracted from the first image. More specifically, from the luminosity of each pixel 1 of the first image is subtracted the luminosity of the corresponding pixel 2 in the second image. In the case where the result of the difference between two pixels in absolute value is less than a sensitivity threshold, the latter is set to zero. A resulting image is thereby obtained in which the pixels, the luminosity of which is different from zero, correspond to portions of the earthworm having moved between both images. By carrying out a sweep of the pixels of the resulting image, the number of pixels 3 for which luminosity has changed between both analyzed images is determined. This allows characterization of the movement of the earthworm in the container between two successive analyzed images.

Within the scope of the present invention, by <<corresponding pixel>> is meant a pixel for which the coordinates (x2, y2) in a second image are the same as the coordinates (x1, y1) of a pixel in a first image (i.e. x1=x2 et y1=y2).

Advantageously, the sensitivity threshold allows suppression of the acquisition noises. Indeed, even without any movement of the earthworm, the luminosity of two corresponding pixels in the first and second images may change because of acquisition noise.

Once the number of pixels is known for which luminosity has changed between both images, the type of response generated by the product is determined 50, 60. To do this, the number of pixels for which luminosity has changed is compared 50 with a threshold called a <<hyperactivity threshold>>. If this number of pixels is greater than the hyperactivity threshold, then the product has an irritating effect. This irritation is all the more marked that the time spent above the hyperactivity threshold during the experiment may be significant.

In an alternative embodiment of the method according to the invention, the number of pixels for which luminosity has changed between both images is also compared with another threshold called a <<hypoactivity threshold>>. If this number of pixels is less than the hypoactivity threshold, then the product has an antalgic and/or anesthetic effect all the more marked that the time spent below the hypoactivity threshold during the experiment may be significant.

Advantageously, the determination of eye or skin discomfort is a function of time. With this, it is possible to quantify the eye or skin discomfort associated with the product. This quantification of the eye discomfort is characterized by the hyperactivity (or hypoactivity) duration of the earthworm during the experiment on the one hand, and by the moment when this hyperactivity (or hypoactivity) occurs on the other hand.

In order to explain this phenomenon, let us only consider the irritating effect of a product. As regards the duration of hyperactivity, it is understood that the longer the earthworm spends time in hyperactivity, the greater is the discomfort (eye or skin discomfort) as regards the moment of this hyperactivity, the shorter the duration between the deposition of the product in the container and the starting point of the hyperactivity, the more significant the (eye or skin) discomfort of the product. On the contrary, if the hyperactivity of the earthworm occurs 14 minutes after the beginning of the experiment, it may be considered that the product has a lesser irritating effect (relatively to a product for which the worm becomes hyperactive as soon as it is placed in contact with the latter).

Table 1 gives a list of results from the measurement on non-irritating products such as pure water or a perfect collyrium followed by an active ingredient, the irritating nature of which is increasing.

TABLE 1

| Hyperactivity time (s) | 60 | 120 | 180 | 240 | 300 | 360 | 420 | 480 | 540 | 600 |
|---|---|---|---|---|---|---|---|---|---|---|
| Water | 0 | 0 | 0 | 0 | 0 | 0 | 0 | 0 | 0 | 0 |
| Collyrium 1 | 0 | 0 | 0 | 0 | 0 | 0 | 0 | 0 | 0 | 0 |
| Active ingredient 1 | 0 | 0 | 0 | 0 | 0 | 2 | 3 | 3 | 2 | 0 |
| Active ingredient 2 | 0 | 0 | 0 | 5 | 11 | 6 | 3 | 0 | 0 | 0 |
| Active ingredient 3 | 0 | 0 | 5 | 9 | 9 | 2 | 0 | 0 | 0 | 0 |
| Active ingredient 4 | 0 | 5 | 12 | 13 | 10 | 5 | 0 | 0 | 0 | 0 |

Each line indicates for a given product the time spent in hyperactivity for a period of 60 seconds and this for a total of 10 mins.

Curve 1 gives a time representation of this table.

Table 2 repeats the total duration in hyperactivity over 10 minutes of the previous products and also indicates the latency of the first hyperactivity.

TABLE 2

| Product | Hyperactivity time | Hyperactivity latency 1 |
|---|---|---|
| Water | 0 | 600 |
| Collyrium 1 | 0 | 600 |
| Active ingredient 1 | 10 | 320 |
| Active ingredient 2 | 25 | 220 |
| Active ingredient 3 | 25 | 160 |
| Active ingredient 4 | 45 | 100 |

The active ingredients 2 and 3 have an equivalent global duration but the active ingredient 3 is classified as being more irritating since it causes a faster response.

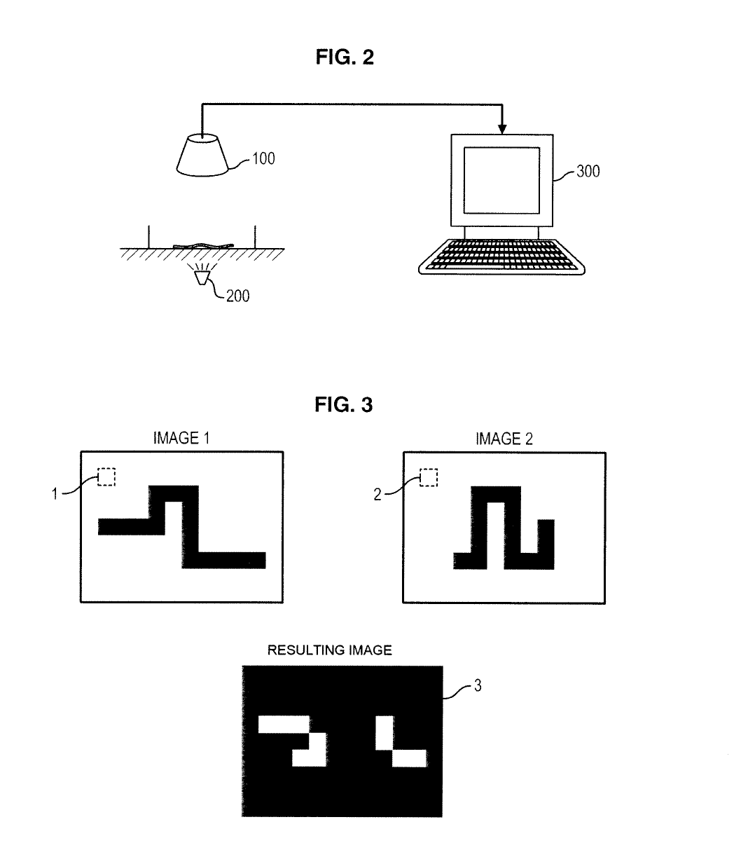

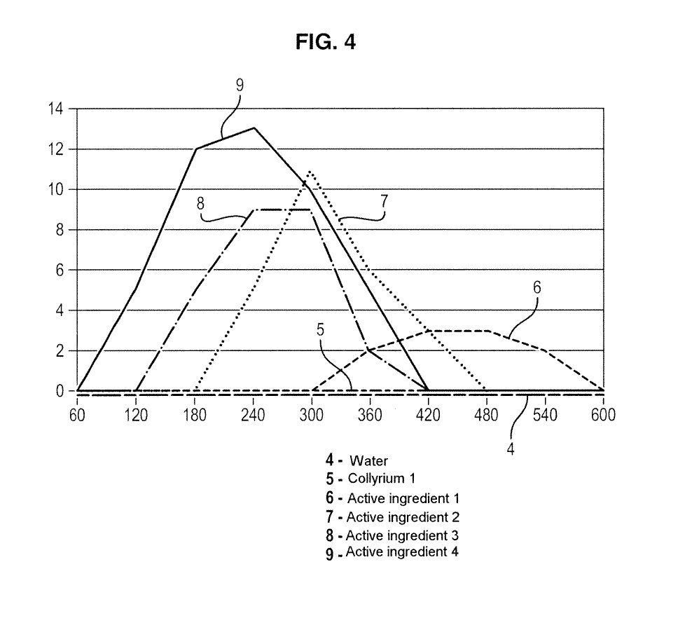

An embodiment of the device according to the invention will now be described with reference to FIG. 2. The device according to the invention comprises acquisition means 100. The acquisition means for example comprise a CCD type camera. The acquisition means 100 allow acquisition of images at a certain frequency (for example 25 images per second) and all along the experiment (for example 10 minutes).

Depending on the aperture of the CCD camera, it is possible to acquire an image representing several containers each comprising an earthworm. In this case, each container may be associated:
with a respective product for comparing the irritating (or antalgic/anesthetic) effect of different products, or
with the same product in different concentrations in order to compare the irritating (or antalgic/anesthetic) effect of a product depending on its concentration.

In order to increase the relevance of the comparison, earthworms are used having the same characteristics, in terms of species, weight, size and age—from one container to the other.

In the case when several containers are illustrated in a same image, the latter is subdivided into a plurality of regions which are processed independently of each other, each region corresponding to a respective container.

The acquisition means 100 are associated with a light source 200 in order to illuminate the object to be imaged. In an embodiment, the light source 200 comprises a viewing box on which is positioned the container containing the earthworm. This allows the contrast between the earthworm and the container in which it is contained to be increased. However, the viewing box has the drawback of heating. This may be a nuisance during the analysis of the volatile product, the concentration of which may change because of the evaporation generated by the heat produced by the viewing box. Moreover, in certain cases, the irritating effect may be increased (or minimized) because of the heat generated by the viewing box. This is why in an embodiment, the light source is a source of infrared light positioned under the container containing the earthworm. The infrared light source has the advantage of generating much less heat than a viewing box and of avoiding modification of the photosensitive active ingredients. A combination of a cold source of white light and infrared light which may be used independently further gives the possibility of widening the field of applications.

The acquisition means 100 are connected to processing means 300 for processing the acquired images. The processing means 300 for example comprise a microprocessor which may be integrated to the acquisition means 100, or be distant from the acquisition means 100. In an embodiment, the processing means 300 comprise a computer of the PC type.

With the processing means 300, it is possible to apply the steps of the method corresponding to the measurement 20, 30, 40 of the movement of the earthworm in the container, and to the determination 50, 60 of the eye discomfort which may be generated by the product according to said measurement.

One skilled in the art will have understood that many modifications may be made to the method and to the device described above without materially departing from the novel teachings shown here. It is therefore quite obvious that the examples which have just been given are only particular illustrations and by no means limiting.

1. A method for identifying a material likely to generate eye or skin discomfort, tingling or irritation, wherein the method comprises the steps:
deposition of a product in a container,
deposition of an earthworm in the container,
measurement of the movement of the earthworm in the container,
determination of the eye or skin discomfort which may be generated by the product according to said measurement.

2. The method according to claim 1, wherein the step for measuring the movement of the earthworm comprises the acquisition of a plurality of images of the earthworm by using image acquisition means, and analysis of the acquired images in order to measure the movement of the earthworm.

3. The method according to claim 2, wherein the analysis of the acquired images comprises the selection of two successive acquired images, the comparison of the luminosity of each pixel of the first image with the luminosity of the corresponding pixel in the second image, the counting of the number of pixels for which luminosity has changed between the first and second images.

4. The method according to claim 1, wherein the step for determining the eye or skin discomfort comprises the estimation of the irritating effect of the product and/or of the antalgic and/or anesthetic effect of the product.

5. The method according to claim 4, wherein the irritating and/or antalgic and/or anesthetic effect of the product is estimated according to the number of pixels for which luminosity has changed between two successively acquired images.

6. The method according to claim 5, wherein the number of pixels for which luminosity has changed between the first and second images is greater than a threshold, then the product has an irritating effect.

7. The method according to claim 5, wherein if the number of pixels for which luminosity has changed between the first and second images is less than a threshold, then the product has an antalgic effect.

8. The method according to claim 1, wherein the determination of the eye or skin discomfort is further time-dependent.

9. The method according to claim 1, wherein it comprises the illumination of the container containing the earthworm by using infrared radiation positioned under the container containing the earthworm.

10. A computer programme product including a programme code recorded on a computer readable medium in order to execute a method for identifying a material likely to generate eye or skin discomfort, tingling or irritation, when the computer programme is applied to a computer in order to be executed, the method comprising the steps of:
deposition of a product in a container,
deposition of an earthworm in the container,
measurement of the movement of the earthworm in the container,
determination of the eye or skin discomfort which may be generated by the product according to said measurement.
